(19) United States
(12) Patent Application Publication
(10) Pub. No.: US 2004/0121331 A1
(43) Pub. Date: Jun. 24, 2004

(54) Methods of amplifying signals in multiplexed protein analysis

(76) Inventor: James A. Apffel JR., Mountain View, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/324,348

(22) Filed: Dec. 19, 2002

(51) Int. Cl. C12Q 1/68

(52) U.S. Cl. 435/6

(57) ABSTRACT

The present invention utilizes nucleic acid fusion proteins as secondary binding agents for detecting protein-protein interactions. The methods involve binding a target protein with a binding agent that is a protein nucleic acid fusion protein, wherein each binding agent has a unique nucleic acid sequence. Once binding of a fusion protein to a target protein occurs, the nucleic acid portion of the fusion protein is amplified and used to probe an array of nucleic acid molecules that are complementary to the amplified nucleic acid portions of the fusion proteins. Detection of the hybridized nucleic acid portion of the array identifies and quantifies the protein-protein interaction.

### BACKGROUND OF THE INVENTION

DNA arrays for multiplexed DNA identification and quantification have revolutionized nucleic acid analysis and molecular biology research. The combination of DNA array technology with amplification methods such as the polymerase chain reaction (PCR) has further allowed the generation of rapid and accurate methods for detecting specific DNA sequences. Similarly, combining DNA array technology with reverse transcription PCR (RT-PCR) has made mRNA expression profiling a unique and convenient method for identifying and quantifying patterns of intracellular transcription of specific genes.

Array technology has also been used to analyze protein-protein interactions. However, the approaches currently available for detection of the protein binding interactions suffer from significant limitations. For example, although in principle surface plasmon resonance and ellipsometry methods should yield both binding kinetic and absolute quantitation data, these techniques lack sensitivity and specificity. Other methods of detecting protein-protein interactions on an array involve labeling a protein ligand with a chemical moiety that generates a signal either before or after capture by a protein on the array. Such moieties include labels such as fluorophores, radioactive labels, and mass tags. The concentration of the protein ligand is typically calculated from the signal intensity of the label. Although useful for samples having high concentrations of protein ligand, the sensitivity of these labeling methods is often insufficient for detecting protein-protein binding interactions when the concentration of the protein ligand is low. The lack of specificity caused by non-specific binding to the array surface further reduces the utility of these assays.

Sandwich assays have also been used in which a second labeled binding agent is employed to detect a second site on a protein of interest. Although these techniques substantially improve the specificity of the detection assay, it's sensitivity remains limited because the signal intensity is still determined by the number of labels that can be attached to the secondary binding agent, as well as the characteristics of the label. The related ELISA assay employs enzyme linked amplification techniques to intensify the detection signal. However, the release of the enzymatically catalyzed products into bulk solution causes diffusion and weakening of the signal.

Several of the advantages of nucleic acid arrays, as they are used to detect nucleic acid binding events, are not present with protein arrays. For example, a low copy number nucleic acid can be amplified before application to an array in order to improve the likelihood of detecting a ligand binding to it. Such amplification can also be employed to improve the signal to noise ratio by increasing the copy number of a selected nucleic acid molecule as compared with other nucleic acids in a sample. There are no available techniques for amplification of specific proteins within a protein sample, however, so the use of protein arrays to detect protein-protein binding events can be hampered by low concentration of desired binding protein and high background of non-specific binding.

There exists the need for methods of amplifying protein-protein binding event signals on protein arrays, preferably while also decreasing background noise. The present invention utilizes DNA array and amplification technology to detect protein-protein interactions.

### SUMMARY OF THE INVENTION

The present invention provides a sandwich assay approach for identifying protein-protein interactions in which a primary capture agent is utilized to anchor a target protein to the surface of a solid support, and a detectable binding agent is employed to bind to the target protein. In preferred embodiments of the invention, the binding agent includes a protein portion and a nucleic acid portion. For example, the protein portion of the binding agent may be a protein or protein fragment that is being tested for its ability to bind to a selected target. The nucleic acid portion may be a unique sequence that represents the protein portion of the binding agent. Binding between the target protein and the binding agent may then be detected by utilizing the nucleic acid portion of the binding agent, which can be amplified to increase the detection signal.

In preferred embodiments of the invention, the nucleic acid portion of the binding agent is amplified to allow more efficient detection of the protein-protein interaction than is allowed by current protein array technology. The amplified nucleic acid proportionately represents the binding agent and can be used ultimately to identify and/or quantify the target protein. According to one preferred embodiment of the invention, once amplified, the nucleic acid portion is applied to a spatial array of nucleic acid molecules that are complementary to the nucleic acid portions of the one or more binding agents used in the binding assay. In certain preferred embodiments, the number of complementary nucleic acids on the array represents the number of binding agents used in the binding assay. Hybridization of an amplified nucleic acid portion to a complementary nucleic acid thereby identifies and/or quantifies the target protein-binding agent interaction. In certain preferred embodiments, detection is carried out using a label that is incorporated into a nucleic acid portion during the amplification process. However, it will be appreciated that a variety of available detection methods may be used, including those described herein.

According to the present invention, the nucleic acid portion of the binding agent may be either DNA or RNA. In one preferred embodiment, the nucleic acid portion of the binding agent is DNA. In another preferred embodiment, the nucleic acid portion of the binding agent is RNA. In particularly preferred embodiments, the nucleic acid portion of the binding agent is a messenger RNA that encodes the amino acid sequence of the protein portion of the binding agent. In certain preferred embodiments, the protein portion of the binding agent is a direct translation product of the mRNA portion of the binding agent.

### BRIEF DESCRIPTION OF THE DRAWINGS

FIG. 1 is a flow chart illustrating one embodiment of the method of the invention, which includes amplification and labeling of a nucleic acid using the polymerase chain reaction and application of the amplified nucleic acid to a DNA array.

### DEFINITIONS

The term “background” or “background noise” means any signal other than a signal generated by the binding of a ligand to an intended target. Those of ordinary skill in the art will appreciate that binding assays are generally done (and is certainly so in the array work described herein) in the presence of alternative compounds that show some chemical similarity with the target. The non-specific binding or “sticking” of such alternative compounds creates background signals. For example, in protein arrays, proteins that are not a ligand of the intended target may adhere non-specifically to the target protein to create background signals. On nucleic acid arrays, nucleic acid ligands having less than 100% complementarity to the target nucleic acid may hybridize to the intended target to generate background signals. For either nucleic acids or proteins, incomplete removal of a non-specific ligand from an intended target may increase background noise. Also, to the extent binding is detected indirectly (e.g., by quantifying fluorescence), there may be some signal from a source other then a marker on a capture agent. For example, background fluorescence may also be produced by intrinsic fluorescence of the array or the fluorescent components themselves. Various methods are available for detecting and quantifying background signals. For example, background may be calculated as the average signal intensity produced by binding to targets that are specific for any ligand found in the sample. Background signal intensity can also be calculated as the average signal intensity produced by regions of the array that completely lack any target.

A “binding agent” is a protein-nucleic acid fusion molecule, natural or synthetic, in which the protein portion binds to a target protein in the inventive binding assay. The nucleic acid portion of the binding agent is double-stranded or single-stranded DNA or RNA. This nucleic acid portion can include natural, modified, or synthetic nucleotides. In certain preferred embodiments, the nucleic acid portion comprises mRNA. According to this embodiment, the protein portion of the binding agent is preferably a translation of the mRNA portion of the binding agent. The unique sequence of the mRNA portion thereby identifies the protein portion of the binding agent. mRNA fusion proteins are well known in the art, as described herein. Where the nucleic acid portion of the binding agent is DNA, the DNA is a unique sequence that differentiates the protein portion of the binding agent from other protein portions of other binding agents. In certain preferred embodiments, the DNA encodes the protein portion of the binding agent.

A “capture agent” is a natural or synthetic chemical entity, preferably a polypeptide molecule that binds to a target protein at a different site than the binding agent. In certain preferred embodiments, the capture agents of the present invention are bound to a solid support, e.g., an affinity column, magnetic bead etc. via a chemical bond. Preferably the capture agent is bound to a solid support via a covalent or non-covalent bond. The capture agents can be associated with the solid phase with or without a bound target protein. In certain preferred embodiments, the capture agent is associated with the solid phase prior to binding to a target protein. In other preferred embodiments, the capture agent is associated with the solid phase after binding to a target protein

As used herein, the term “hybridize” refers to the interaction between two nucleic acid strands and will typically be modified with either “under low stringency,” “under medium stringency,” “under high stringency,” or “under very high stringency” conditions. Hybridization conditions of varying stringency are well known in the art and can be found, for example, in Current Protocols in Molecular Biology (1989) John Wiley & Sons, N.Y., 6.3.1-6.3.6, which is incorporated by reference. Aqueous and nonaqueous methods are described in that reference and either can be used. The following are non-limiting examples of specific hybridization conditions referred to herein: 1) low stringency hybridization conditions: 6× sodium chloride/sodium citrate (SSC) at about 45° C., followed by two washes in 0.2× SSC, 0.1% SDS at least at 50° C. (the temperature of the washes can be increased to 55° C. for low stringency conditions); 2) medium stringency hybridization conditions: 6× SSC at about 45° C., followed by one or more washes in 0.2× SSC, 0.1% SDS at 60° C.; 3) high stringency hybridization conditions: 6× SSC at about 45° C., followed by one or more washes in 0.2× SSC, 0.1% SDS at 65° C.; and preferably 4) very high stringency hybridization conditions: 0.5M sodium phosphate, 7% SDS at 65° C., followed by one or more washes at 0.2× SSC, 1% SDS at 65° C. Very high stringency conditions are the preferred conditions and the ones that should be used unless otherwise specified (Sambrook et al., Molecular Cloning. A Laboratory Manual, Chapter 15, Cold Spring Harbor Press, New York, 2^{nd} ed. (1989), incorporated herein by reference).

The phrase “hybridizes specifically to” means interacts preferentially with a target nucleic acid molecule under the conditions of the reaction. The present invention contemplates reactions in the presence of complex competitors. It is generally recognized that increasing the temperature or decreasing the salt concentration of the buffer containing the nucleic acids are denaturing events. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization occurs only when mismatches are minimized. The stability of duplexes formed between RNAs and/or DNAs are generally in the order of DNA:DNA >RNA:DNA>RNA:RNA, in solution. Long complementary nucleic acids have better stability with a nucleic acid, such as the nucleic acids amplified from the binding agents of the present invention, but poorer mismatch discrimination than shorter complementary nucleic acids. (Mismatch discrimination refers to the measured hybridization signal ratio between a perfect match complementary nucleic acid and a single base mismatch complementary nucleic acid). Shorter complementary nucleic acids (e.g., 8-mers) discriminate mismatches very well, but the overall duplex stability is low.

By “substantially purified” is meant that a protein (or nucleic acid) has been separated from at least some of the components with which it associates when it is first generated, e.g., either in nature or recombinantly. Typically, a protein (or nucleic acid) is substantially pure when it is at least 60%, by weight, free from the proteins and naturally occurring organic molecules with which it is associated when it is first generated. Preferably, a protein (or nucleic acid) is substantially pure when it is at least 70%, even more preferably 75%, 80%, 85%, 90%, 95%, by weight, free from the proteins and naturally occurring organic molecules with which it is associated when it is first generated. Most preferably, a protein (or nucleic acid) is substantially pure when it is at least 99%, by weight, free from proteins and naturally occurring organic molecules with which it is associated when it is first generated. Purity can be measured by any appropriate method, e.g., by column chromatography, polyacrylamide gel electrophoresis, or HPLC analysis.

A “target protein” is a protein for which protein-protein binding interactions are assessed. Preferably, the target protein is at least 30 amino acids in length, preferably 50 amino acids in length. However, unique peptides of seven to nine amino acids could also be target proteins of the invention. The target proteins of the present invention can be components of complex mixtures of proteins or complex mixtures of proteins and nucleic acid, for example, complex mixtures from a naturally occurring or recombinant source. In certain preferred embodiments, the target proteins can be substantially purified from a complex mixture (e.g., at least 60% pure, at least 70%, at least 80%, at least 90%, at least 95%, at least 97%, at least 98%, or greater than 99%, pure by weight). In other preferred embodiments, the target protein is not purified from a complex mixture of proteins, but is tested in the context of the complex mixture of proteins. Other target proteins are chemically synthesized by methods standard in the art. It is a goal of the present invention to identify proteins that are binding partners to the target proteins of the invention.

### DETAILED DESCRIPTION OF CERTAIN PREFERRED EMBODIMENTS OF THE INVENTION

The present invention provides a system for detecting protein-protein interactions that utilizes binding agents, which include a protein portion that binds to a target protein and a nucleic acid portion that, for example, serves as an identifier of the protein portion and/or binding. According to the invention, each binding agent has a unique nucleic acid sequence that may be used to distinguish the protein portion of one binding agent from the protein portions of other binding agents. In particularly preferred embodiments, the sequence of the nucleic acid portion reflects the binding agent's primary amino acid sequence.

In one simple embodiment, the present invention provides methods in which a binding agent comprising a protein portion and a nucleic acid portion is contacted with a target protein for a time sufficient to allow binding of the protein portion to the target protein. Once bound, the nucleic acid portion of the binding agent is detected. One advantage of the present invention is that, prior to detection, the nucleic acid portion of the binding agent can be amplified. This feature increases the overall sensitivity of the detection assay, solving the problem of decreased sensitivity that arises when the concentration of a target protein is low. As mentioned above, complex biological samples, such as those generated from tissues or cells or even purified protein samples, often contain a limited quantity of target protein. Previously available assays for detecting protein-protein interactions involving the relevant protein lacked a means by which to significantly amplify the detection signal. The present invention combines protein-protein binding technology with amplification and DNA array technology to accurately detect protein-protein interactions.

The present invention further provides capture agents that also bind to the target proteins. In preferred embodiments, the capture agent binds to the same target protein as the binding agent. According to the present invention, the capture agent and the binding agent preferably bind to different sites on the target protein. This prevents competition between the two molecules for the same site on the target (e.g., inhibition of binding of the binding agents by the capture agent and vice versa). Spatial separation between the sites promotes binding of both the binding agent and the capture agent to the target simultaneously so that a ternary capture agent-target-binding agent complex is formed. Spatial separation may be even more critical to detecting binding between a binding protein and a capture agent bound target if one or both of these molecules is labeled with a bulky chemical moiety that would further spatially inhibit binding.

The capture agents of the invention function to bind the target protein to the surface of a solid support. The solid support provides a stable attachment surface for the target protein so that the binding agents can be contacted with the target protein for a time sufficient to allow binding. Binding agents that do not specifically bind to the target can subsequently be separated from the target protein. Separation of bound from unbound binding agent increases the sensitivity of the detection method. The greater the separation of bound from unbound binding agent, the greater the decrease in background noise in the detection step.

Those skilled in the art will further appreciate that any of a variety of means known in the art for separating the capture agent-target-binding agent complexes from unbound binding agents can be used in the present invention, but typically include a solid support. In certain preferred embodiments, the solid support is a bead, such as a magnetic bead. In other preferred embodiments, the solid support is an array or a column. Columns that selectively bind the binding agent on an immobilized target can be used to partition the bound and unbound binding agents. Alternatively, a cell membrane or cell membrane fragment having the target on its surface can bind the binding agents and be used to separate out target-bound binding agents from the mixture of binding agents. The choice of separating method will depend on the properties of the target and of the target-binding agent complex or the ternary capture agent-target-binding agent complex and can be made according to principles and properties known to those of ordinary skill in the art. Those skilled in the art will appreciate that any known solid support can be employed in the present invention.

In preferred embodiments, once a binding agent is bound to a target protein, the nucleic acid portion of the binding agent is detected using any method available in the art. In particularly preferred embodiments, the detection includes amplification. Typically, nucleic acids are amplified using the polymerase chain reaction (PCR). In order to perform the amplification, primer pairs may be generated that are specific to the nucleic acid portion of the binding agent. For assays that include more than one binding agent (the binding agents each having a different protein portion and a different nucleic acid identifier), the nucleic acid portions may include common 3′ and/or 5′ portions so that a single set of forward and reverse primers can be used to amplify all of the different nucleic acid portions of the binding agents. Alternatively, unique primer binding sites can be included in each nucleic acid portion, requiring a unique primer pair for each binding agent. The “uniqueness” of the primer binding sites can be adjusted by their sequence or by the experimental conditions used for hybridization, or both.

According to the present invention, once a binding agent is bound to a target protein and the unbound proteins may be separated from the bound proteins, the nucleic acid portions may be detected based on any feature. In certain preferred embodiments, the nucleic acid portions are detected based on their nucleotide sequence. Any of a wide variety of methods may be used to sequence the nucleic acid portion of a binding agent. In other preferred embodiments, the nucleic acid portions of the binding agent may be detected by size. Nucleic acid portions of different size might be identified by any method available in the art, for example, without limitation, electrophoretic polyacrylamide gel electrophoresis, column chromatography, etc. The nucleotide sequence and/or size of a nucleic acid portion may also be determined by analyzing a restriction enzyme digest of the amplified nucleic acid portion.

In other preferred embodiments, the DNA portion of a capture agent is detected by 1) absorbing the target protein onto a solid support or particle; 2) deactivating the surface of the solid support or particle; 3) binding a capture agent/DNA complex to the target protein on the solid support or particle; 4) washing the surface of the solid support of particle to remove unbound capture agent/DNA complex; 5) release the capture agent/DNA complex from the surface of the solid support or particle; 6) amply the DNA portion of the capture agent/DNA complex; 7) identify and/or quantify the amplified DNA on a DNA array.

In yet other preferred embodiments, the nucleic acid portion is detected by the presence of a label. According to the present invention, the labels can be used to 1) detect a protein-protein binding interaction; 2) identify a protein-protein binding interaction; or 3) quantify a protein-protein binding interaction. For example, according to the present invention, a label can be used to detect the presence of a specific binding agent-target interaction, to identify the members of a binding agent-target interaction, or to quantify a binding agent-target interaction.

In certain preferred embodiments, a unique label is incorporated onto the nucleic acid portion of the binding agent during the amplification. For example, the amplification can be done with a labeled primer so that each amplified copy of a nucleic acid portion has an incorporated label that generates a specific signal. Assuming that all of the sequences amplify equally, the concentration of the original population of nucleic acid portions will be represented proportionately, albeit geometrically multiplied. Alternatively, the label may be added to the nucleic acid portion subsequently to the amplification process. As will be appreciated by those skilled in the art, a variety of labels for nucleic acids are readily available, including those described herein.

In related embodiments, the nucleic acid portion of the binding agent may be detected by combining the methods of amplifying the nucleic acid portions with nucleic acid array technology. According to the present invention, the nucleotide sequence of an amplified nucleic acid portion can be identified by hybridization to its complementary strand on a spatial array of nucleic acids. For example, each nucleic acid on the array may be complementary to a nucleic acid portion of a different binding agent. The sequence of the amplified nucleic acid portion may be identified by the known position of its complement immobilized on the array.

In preferred embodiments, arrayed complementary nucleic acids may be contacted with amplified nucleic acid(s) for a time and under conditions sufficient for the amplified nucleic acid portions to hybridize to their respective complementary nucleic acids on the array. Once unbound amplified nucleic acid portions are removed from the array, e.g., by washing, the amplified nucleic acid portions bound to the array may be detected. In preferred embodiments, the amplified nucleic acid portions bound to the array are detected by using a label. As described above, in certain preferred embodiments, the amplified nucleic acid portions bound to the array are identified spatially, based on the known position of the complementary strand and by the signal provided by the label. In such embodiments, the same label may be used for each different nucleic acid portion. Alternatively, a unique label can be employed to identify each different nucleic acid portion.

Where a unique label is employed to identify nucleic acid portions hybridized to an array, the choice of label further depends on the number of different amplified nucleic acid portions being assessed. For example, if only one nucleic acid portion were being assessed in the protein-protein binding assay, only a single unique label would be required. If more than one nucleic acid portion were being assessed in the protein-protein binding assay, it might be appropriate for a different label to be used for each binding agent. For example, in certain preferred embodiments, different color fluorescent labels might be used for each binding agent used in the assay.

The choice of label may further depend on whether the assay is used to identify or to quantify the nucleic acid portion. As described above, for identification, the same label can be used for each different binding agent or unique labels may be employed. For quantification, however, it might be preferable to use the same label for each amplified nucleic acid portion to eliminate any differences in signal intensity due to differences in the label. According to this aspect of the present invention, nucleic portions linked to the same label may be quantified by hybridizing them to a spatial array of complementary nucleic acids and detecting the signal generated by the label. It will be appreciated by those skilled in the art that different labels can be used for quantification purposes if, e.g., they generate the same signal intensity or if differences in signal intensity are accounted for. Either of these tactics, and others known in the art, would reduce or eliminate errors in quantification that would be due to differences in the labels themselves.

According to preferred embodiments, each nucleic acid portion of a binding agent that is bound to a target protein is amplified equally well to achieve quantitative results. This allows the amount of labeled nucleic acid portion bound to the array to remain proportional to a) the amount of binding agent bound to the target protein, and b) the amount of target protein itself. Ultimately, hybridization of the appropriate amplified nucleic acid portion to it's complementary strand on the array identifies and quantifies the binding agent participating in the target-binding agent interaction as well as the target protein.

As would be appreciated by those skilled in the art, whether detection of the nucleic acid portion is carried out by sequence analysis, size determination, or labeling of the nucleic acid portion, in order to quantify a test sample, the amount of a test sample is typically compared to the amount of a control sample. In the present invention, the test sample is the amplified nucleic acid portion and the control sample is a specific quantity of a co-amplified oligonucleotide. Of course, more than one control sample may be used. Those skilled in the art will appreciate the myriad of ways that a control sample may be used to quantify a test sample.

In related embodiments, identification and quantification can occur simultaneously on the array. For example, the quantity of an amplified nucleic acid portion on an array can be determined by the signal intensity of the label. Simultaneously, the identity of the amplified nucleic acid portion can be determined by the identity of the label, or alternatively, the position of the hybridized nucleic acid portion on the array. However, the type of label used in such a dual-type assay would preferably be a single type of label for the multiple different binding agents.

Multiple Capture Agents and Targets

In certain preferred embodiments, capture agents are spatially arrayed on the surface of a solid support so that the position of the capture agent can be used to identify the capture agent. Wherein a binding agent-target complex is known, additional partners that bind to the binding agent-target complex can be screened. The additional partners can be screened as capture agents on a spatial array. In preferred embodiments, one or both of the binding agent and target may be labeled so that interaction with the capture agent may be readily detected. In certain preferred embodiments, where multiple targets or multiple binding agents are used, unique labels can be used to identify each particular member of the complex formed. For example, each different binding agent or target may have a different color fluorescent label to identify its binding to a particular capture agent on the array.

In other preferred embodiments, multiple different capture agents are used in a binding assay with one or more different targets and/or one or more different binding agents. For example, if the identity of the capture agent is spatially encoded on an array of capture agents and the binding agents are labeled with unique labels, the identity of both the capture agent and the binding agent in a ternary capture agent-target-binding agent complex can be determined. That is, the unique labels on the binding agent can be used to identify the binding agents and the signals generated by the labels can further are used to locate the position of the bound capture agent on the capture agent array, thereby identifying the capture agent. The identity of the target may be known if only a single target is used. Alternatively, if a particular partnership between a target and capture agent or target and binding agent is known, the identity of the target can be based on the identity of the capture agent or the binding agent in the complex.

In alternative embodiments, once the binding agent is bound to an array of capture agents, labeled oligonucleotide probes that are complementary to the nucleic acid portions of the binding agents can be used to probe the surface of the chip. Specific hybridization between the probe and the nucleic acid portion of the binding agent may serve to identify the binding agent bound to the target molecule. In related embodiments, a capture agent is bound to an array of binding agents. Labeled oligonucleotide probes that are complementary to the nucleic acid portions of the capture agents can be used to probe the surface of the chip. Either the target protein or the capture agent can be used as the bait in the present invention. The levels of the binding agent or capture agent can be optimized to maximize performance.

Complex Biological Sample

Complex biological samples from which the target proteins of the present invention may be typically derived from (“derived from” meaning originating from a sample from nature) include physiological sources. The physiological source may be a variety of eukaryotic sources, with physiological sources of interest including sources derived from single-celled organisms such as yeast and multicellular organisms, including plants and animals, where the physiological sources from multicellular organisms may be derived from particular organs or tissues of the multicellular organism, or from isolated cells derived therefrom.

For example, the target proteins may be based on genes obtained or derived from naturally occurring biological sources, particularly mammalian sources and more particularly mouse, rat or human sources, where such sources include: fetal tissues, such as whole fetus or subsections thereof, e.g. fetal brain or subsections thereof, fetal heart, fetal kidney, fetal liver, fetal lung, fetal spleen, fetal thymus, fetal intestine, fetal bone marrow; adult tissues, such as whole brain and subsections thereof, e.g. amygdala, caudate nucleus, corpus callosum, hippocampus, hypothalamus, substantia nigra, subthalamic nucleus, thalamus, cerebellum, cerebral cortex, medula oblongata, occipital pole, frontal lobe, temporal lobe, putameri, adrenal cortex, adrenal medula, nucleus accumbens, pituitary gland, adrenal gland and subsections thereof, such as the adrenal cortex and adrenal medulla, aorta, appendix, bladder, bone marrow, colon, colon proximal with out mucosa, heart, kidney, liver, lung, lymph node, mammary gland, ovary, pancreas, peripheral leukocytes, placental, prostate, retina, salivary gland, small intestine, skeletal muscle, skin, spinal cord, spleen, stomach, testis, thymus, thyroid gland, trachae, uterus, uterus without endometrium; cell lines, such as breast carcinoma T-47D, colorectal adenocarcinoma SW480, HeLa, leukemia chronic myelogenous K-562, leukemia lymphoblastic MOLT-4, leukemia promyelocytic HL-60, lung carcinoma A549, lumphoma Burkitt's Daudi, Lymphoma Burkitt's Raji, Melanoma G361, teratocarcinoma PA-1, leukemia Jurkat; and the like. Where the target proteins are derived from naturally occurring sources, such as mammalian tissues, as described above, the target proteins may be derived from the same or different organisms, but will usually be derived from the same organism. In addition, the target proteins on the array can be derived from normal and disease or condition states of the same organism, like cancer, stroke, heart failure; aging, infectious diseases, inflammation, exposure to toxic, drug or other agents, conditional treatment, such as heat shock, sleep deprivation, physical activity etc., different developmental stages, and the like.

In obtaining the target protein to be analyzed from the physiological source from which it is derived, the physiological source may be subjected to a number of different processing steps, where such processing steps might include tissue homogenization, cell isolation and cytoplasm extraction, nucleic acid extraction and the like, where such processing steps are known to those of skill in the art. Methods of isolating proteins from cell, tissues, organs, or whole organisms are known to those of skill in the art and are described in Maniatis et al. (1989), Molecular Cloning. A Laboratory Manual 2^{nd} Ed. (Cold Spring Harbor Press) or Ausubel et al., Current Protocols in Molecular Biology, John Wiley & Sons and Green Publishing Company, 1994.

Binding Agents

The binding agents of the present invention preferably are (covalent or non-covalent) fusions between a protein or protein fragment of interest and a unique nucleic acid molecule. The nucleic acid portion of the binding agent may be either a DNA molecule or an RNA molecule. Preferably, the nucleic acid portion of the fusion protein identifies the protein portion. For example, the sequence of the nucleic acid portion may encode the protein portion. Alternatively, the sequence of the nucleic acid portion may encode a fragment of the protein portion that differentiates the protein portions from one another. In yet other preferred embodiments, the sequence of the nucleic acid portion does not encode the protein portion, or a fragment of the protein portion, but is simply a unique sequence compared to the nucleic acid portions of other binding agents. Whether or not the nucleic acid portion encodes the protein portion, the nucleic acid portion can hybridize to a specific complimentary sequence in an array type assay.

RNA portions of binding agent molecules of the invention include those derived from total RNA, polyA^{+RA}, polyA^{-RNA}, snRNA (small nuclear), hnRNA (heterogeneous nuclear), cytoplasmic RNA, pre mRNA, mRNA, cRNA (complementary), and the like. In particularly preferred embodiments, the nucleic acid portion of the binding agent is a messenger RNA (mRNA) molecule. Preferably, the mRNA-protein fusions include a protein portion covalently linked to its own messenger RNA. These mRNA-protein fusions are synthesized by in vitro or in situ translation of mRNA molecules containing a peptide acceptor attached to their 3′ ends (see, e.g., PCT/US98/00807, incorporated herein by reference). In one preferred embodiment, after readthrough of the open reading frame of the message, the ribosome pauses when it reaches the designed pause site, and the acceptor moiety occupies the ribosomal A site and accepts the nascent peptide chain from the peptidly-tRNA in the P site to generate the RNA-protein fusion. The covalent link between the protein and the mRNA (in the form of an amide bond between the 3′ end of the mRNA and the C-terminus of the protein that it encodes) allows the genetic information in the protein to be recovered and amplified, e.g., by PCR, following reverse transcription of the RNA. Once the fusion is generated, it may be used as a binding agent to assess the ability of the protein portion to bind to a target protein.

One method of generating mRNA protein fusions as binding agents utilizes puromycin. Puromycin is a very powerful antibiotic inhibitor of cell growth due to its ability to block polypeptide of chain elongation. Structurally, it is an analog of the 3′ end of aminoacyl-tRNA, and thus is readily capable of entering the ribosomal A site to be transferred to nascent polypeptide chains by peptidyl transferase. Covalent bonding of puromycin to the C-terminal end of an encoded protein in a cell-free translation system using rabbit reticulocyte lysates is one method of generating mRNA fusion proteins (Nemoto et al., FEBS Letters 414 (1997) 405-408, incorporated herein by reference). Other in vitro translation systems for generating mRNA protein fusions using puromycin termination are available in the art (see, for example, Miyamoto-Sato et al., Nucleic Acids Research, (2000) Vol. 28, No 5, 1176-1182; and Roberts and Szostak, Proc. NatL. Acad. Sci., USA (November 1997) 94:12297-12302, each incorporated herein by reference). One of the most attractive features of puromycin is the fact that it forms a stable amide bond to the growing peptide chain, thus allowing for more stable fusions than potential acceptors that form unstable ester linkages. In particular, the peptidyl-puromycin molecule contains a stable amide linkage between the peptide and the O-methyl tyrosine portion of the puromycin. The O-methyl tyrosine is in turn linked by a stable amide bond to the 3′-amino group of the modified adenosine portion of puromycin. One exemplary synthesis of puromycin is described in PCT/US98/00807, incorporated herein by reference.

Other choices for acceptors include tRNA-like structures at the 3′ end of the mRNA, as well as other compounds that act in a manner similar to puromycin. Such compounds include, without limitation, any compound which possesses an amino linked to an adenine or an adenine-like compound, such as the amino acid nucleotides, phenylalanyl-adenosine (A—Phe), tyrosyl adenosine (A—Tyr), and alanyl adenosine (A—Ala), as well as amide-linked structures, such as phenylalanyl 3′ deoxy 3′ amino adenosine, alanyl 3′ deoxy 3′ amino adenosine, and tyrosyl 3′ deoxy 3′ amino adenosine; in any of these compounds, any of the naturally-occurring L-amino acids or their analogs may be utilized. In addition, a combined tRNA-like 3′ structure-puromycin conjugate may also be used in the invention.

As a step toward generating RNA-protein fusions, the RNA portion of the fusion may be synthesized. This may be accomplished by direct chemical RNA synthesis or, more commonly, is accomplished by transcribing an appropriate double-stranded DNA template. Such DNA templates may be created by any standard technique, including any technique of recombinant DNA technology, chemical synthesis, or both, see Ausubel et al., Current Protocols in Molecular Biology, John Wiley & Sons and Green Publishing Company, 1994; Sambrook et al., Molecular Cloning: A Laboratory Manual, Chapter 15, Cold Spring Harbor Press, New York, 2^{nd} ed. (1989), incorporated herein by reference).

The RNA portion of an RNA-protein fusion may be chemically synthesized using standard techniques of oligonucleotide synthesis. Alternatively, as longer RNA sequences are utilized, the RNA portion may be generated by in vitro transcription of a DNA template. In one preferred approach, T7 polymerase is used to enzymatically generate the RNA strand. Other appropriate RNA polymerases for this use include, without limitation, the SP6, T3 and E. coli RNA polymerases (Ausubel et al., supra). In addition, the synthesized RNA may be, in whole or in part, modified RNA.

In generating mRNA-protein fusions, puromycin (or any other appropriate acceptor peptide) may be covalently bonded to the template sequence. This step may be accomplished using T4 RNA ligase to attach the puromycin directly to the RNA sequence, or preferably the puromycin may be attached by way of a DNA “splint” using T4 DNA ligase or any other enzyme that is capable of joining together two nucleotide sequences (Ausubel et al., supra). rRNA synthetases may also be used to attach puromycin-like compounds to RNA, for example, phenylalanyl tRNA molecules containing a 3′ amino group, generating RNA molecules with puromycin-like 3′ ends (Fraser and Rich, Proc. Natl Acad. Sci. USA (1973) 70:2671, incorporated herein by reference). Other peptide acceptors that may be used include, without limitation, any compound that possesses an amino acid linked to an adenine or an adenine-like compound, such as the amino acid nucleotides phenylalanyl—adenosine (A—Phe), tyrosyl adenosine (A—Tyr), and alanyl adenosine (A—Ala), as well as amide-linked structure, such as phenylalanyl 3′ deoxy 3′ amino adenosine, alanyl 3′ deoxy amino adenosine, and tyrosyl 3′ deoxy 3′ amino adenosine; in any of these compounds, any of the naturally-occurring L-amino acids or their analogs may be utilized. A number of peptide acceptors are described, for example, in Krayevsky and Kukhanova, Progress in Nucleic Acids Research and Molecular Biology (1979) 23:1, incorporated herein by reference).

To generate RNA-protein fusions, an in vitro or in situ translation system may be used. Eukaryotic systems are preferred, e.g., the wheat germ and reticulocyte lysate systems, lysates from yeast, ascites, tumor cells (Leibowitz et al., Meth. Enzymol. (1991) 194:536, incorporated herein by reference), and,Yen opus oocyte eggs. However, any translation system that allows formation of an RNA-protein fusion and that does not significantly degrade the RNA portion of the fusion is useful in the invention. Useful in vitro translation systems from bacterial systems include, without limitation, those described in Zubay (Ann. Rev. Genet. (1973) 7:267); Chen and Zubay, Meth. Enzymol. (1983) 101:44; and Ellman (Meth. Etnzytinol. (1991) 202:301, each incorporated herein by reference). In addition, to reduce RNA degradation in any of these systems, protease and nuclease inhibitors, as well as degradation-blocking antisense oligonucleotides specifically hybridize to and cover sequences within the RNA portion of the molecule that trigger degradation (see, e.g., Hanes and Pluckthun, Proc. Nall. Acad. Sci. USA (1997) 94:4937, incorporated herein by reference).

As mentioned above, translation reactions may also be carried out in sitie. In one particular example, translation may be carried out by injecting mRNA into Xenopzis eggs using standard techniques (Capco and Jackle, Dev. Biol., 1982 Nov;94(1):41-50, incorporated herein by reference).

Once generated, RNA-protein fusions may be recovered from the translation reaction mixture by any standard technique of protein or RNA purification. Typically, protein purification techniques are utilized. As shown below, for example, purification of a fusion may be facilitated by the use of suitable chromographic reagents such as dT25 agarose or thipopropyl sepharose. Purification, however, may also or alternatively involve purification based upon the RNA portion of the fusion; techniques for such purification are described, for example in Ausubel et al., supra.

DNA copies of the RNA sequence in the RNA-protein fusion may easily be generated. For example, a DNA copy of a selected RNA fusion sequence is readily available by reverse transcribing the RNA sequence using any standard technique (e.g., using Superscript™ reverse transcriptase). This step may be carried out prior to the binding step, or preferably following that step. According to certain preferred embodiments of the invention, the DNA template is next amplified, either as a partial or full-length double-stranded sequence.

Capture Agents

The capture agents of the present invention may be any agents, natural or synthetic, that bind to a target protein at a different site on the target protein than the binding agent. The capture agents of the present invention link the target molecule(s) to a solid support, e.g., an affinity column, magnetic bead etc. The capture agents are therefore preferably provided on the surface of a solid support. Certain preferred capture agents of the present invention include polypeptide ligands, antibodies, and the like. The capture agents can be modified by any chemical moiety that will perform the function of attaching the capture agent to a solid support. Exemplary modifications include, for example, functionalizing beads or other solid surfaces with cleavable or non-cleavable tags, enzyme/substrate, e.g., biotin/streptavidin, chemical linking moieties capable of binding the surface of a solid support. Linkers and functionalities that are capable of attaching a capture agent to a solid support surface are well known in the art (see, e.g., U.S. Pat. No. 5,789,172, incorporated herein by reference).

Amplification

As described herein, one advantageous feature of the present invention is the ability to amplify the signal that represents a protein-protein interaction by amplifying the nucleic acid portion of the binding agent. The nucleic acid portion of the binding agent can be amplified directly, if it is DNA, or indirectly, e.g., if it is reverse transcribed DNA from an RNA-protein binding agent. Methods of nucleic acid amplification are well known in the art. In general, amplification of a nucleic acid molecule employs a pair of single-stranded oligonucleotide primers together with an enzyme, e.g., a DNA polymerase, which replicates (amplifies) a region of the nucleic acid sample, resulting in multiple copies of the region delimited by the sequences that are complementary to the primers. The pair of primers is chosen so as to amplify a region of the nucleic acid sample containing the unique portion of the nucleic acid that differentiates the binding agents from one another. The size of the region amplified is not critical, but the region must be sufficiently large to include the unique region. The primer pairs can correspond to the unique regions, or alternatively, the primer pairs can correspond to common sequences on either side of the unique region. A high specific binding of the pair of primers to the chosen region is generally accomplished by sufficient complementarity. Strategies for designing and synthesizing primers suitable for amplification of a specific region of a nucleic acid sample are known in the art. As is known in the art, each primer of a pair of amplification primers hybridizes to, and is preferably complementary to, opposite strands of a chosen region. It is preferred that the primers hybridize to a double stranded nucleic acid in locations that are not more than 2 kb apart, are preferably closer together, such as not more than 1kb, 0.5 kb, 0.2 kb, 0.1 kb, 0.01 kb, or 0.0001 kb apart. A suitable DNA polymerase can be used as is known in the art. Thermostable polymerases are particularly convenient for thermal cycling of rounds of primer hybridization, polymerization, and melting. Amplification of single stranded nucleic acids (e.g., RNAs or mRNAs) can also be employed.

One of skill in the art will appreciate that whatever amplification method is used, if a quantitative result is desired, care must be taken to use a method that maintains or controls for the relative frequencies of the amplified nucleic acids to achieve quantitative amplification. Methods of quantitative amplification are well known to those of skill in the art. For example, quantitative PCR may involve simultaneously co-amplifying a known quantity of a control sequence using the same primers used to amplify the nucleic acids of interest. This provides an internal standard that can be used to calibrate the PCR reaction. The nucleic acid array can then include probes specific to an internal standard for quantification of the amplified nucleic acid. Detailed protocols for quantitative PCR are provided in PCR protocols, A Guide to Methods and Applications, Innis et al., Academic Press, Inc. N.Y. (1990).

One example of a quantifying method is to use a confocal microscope and fluorescent labels. For example, The GeneChip™ system (Affymetrix, Santa Clara, Calif.) is one system that is suitable for quantifying hybridizations on arrays. However, it will be apparent to those of skill in the art that any similar system or other effectively equivalent detection method can also be used.

After amplification, it may be desirable to remove and/or degrade any excess primers or nucleotides. This can be done by washing and/or enzymatic degradation, using such enzymes as, for example, endonuclease I or alkaline phosphatase. Other techniques, such as chromatography, magnetic beads, and avidin or streptavidin-conjugated beads, as are known in the art for accomplishing the removal, can also be used. It is not necessary to remove or destroy one of two strands of an amplified DNA product.

Another amplification method that can be used in the present invention is rolling circle amplification, described in U.S. patent application Ser. No. 10/076, 363, filed Feb. 15, 2002, incorporated herein by reference. Rolling circle amplification is particularly applicable to nucleic acids having multiple repeats.

Labeling

According to certain preferred embodiments of the invention, the amplified DNA product is labeled using a template-dependent primer extension reaction prior to its hybridization to complementary oligonucleotides on a solid support. In other preferred embodiments, the protein portion of the binding agent is labeled. In particularly preferred embodiments, the primer molecule contains the label. The primer is hybridized to the denatured amplified double stranded DNA and is extended by one or more labeled nucleotides using, e.g., a mixture of nucleoside triphosphates and a DNA polymerase. Any DNA dependent polymerase can be used in the amplification reaction. These include, but are not limited to E. coli DNA polymerase I, Kienow fragment of DNA polymerase I, T4 DNA polymerase, T7 DNA polymerase, and T. aquatictus DNA polymerase. The extension reaction is preferably performed at the Tₘ of the primer with the template to enhance product formation.

In certain embodiments, the amplified nucleic acid is labeled to provide for detection in the detection step. By “labeled” is meant that the nucleic acid comprises a member of a signal-producing system and is thus detectable, either directly or through combined action with one or more additional members of a signal producing system. As mentioned above, one method of labeling involves using primers that contain the labels, e.g., contain nucleotides that are labeled or are chemically linked to a label on their 5′ or 3′ ends. Alternatively, the label can be covalently attached to the nucleoside triphosphates that serve as reactants for the extension reaction. A typical configuration for carrying out the primer extension step utilizes two different primers that each hybridize to opposite strands of a double stranded DNA.

Examples of directly detectable labels include isotopic and fluorescent moieties incorporated into, usually covalently bonded to, a moiety of the nucleic acid, such as a nucleotide monomeric unit, e.g. dNMP of the primer, or a photoactive or chemically active derivative of a detectable label which can be bound to a functional moiety of the nucleic acid molecule. Isotopic moieties or labels of interest include ³²p, ³³p, ³⁵S, ¹²⁵I, and the like. Enzymes include, e.g. green fluorescent protein, horseradish peroxidase, alkaline phosphatase and others commonly used in an ELSA. Fluorescent moieties or labels of interest include coumarin and its derivatives, e.g. 7-amino-4-methylcoumarin, aminocoumarin, bodipy dyes, such as Bodipy FL, cascade blue, fluorescein and its derivatives, e.g. fluorescein isothiocyanate, Oregon green, rhodamine dyes, e.g. Texas red, tetramethylrhodamine, eosins and erythrosins, cyanine dyes, e.g. Cy3 and Cy5, macrocyclic chelates of lanthanide ions, e.g. quantum dye™, fluorescent energy transfer dyes, such as thiazole orange-ethidium heterodimer, TOTAB, etc.

Alternatively, cleavable mass tags analyzed by Atmospheric Pressure Chemical Ionization (APCI) Mass Spectrometry (MS) may be attached to the amplified nucleic acid portions to detect protein-protein interactions. Using this technique in accordance with the invention, once the target-binding agent complex is formed, the nucleic acid portion of the binding agent is amplified using a primer (forward or reverse) having a cleavable mass tag attached to it. Following amplification, the nucleic acids are separated from the binding complex and hybridized to an array of complementary nucleic acids. The mass tags are then analyzed by FIA-APCI-MS or HPLC-PCI-MS using chemical, thermal, or photolysis to cleave the mass tag from the array. The MS identification and quantification is done on the small mass tag and related back to the initial protein sample.

Labels may also be members of a signal-producing system that acts in concert with one or more additional members of the same system to provide a detectable signal. Illustrative of such labels are members of a specific binding pair, such as ligands, e.g. biotin, fluorescein, digoxigenin, antigen, polyvalent cations, chelator groups and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic product, e.g. alkaline phosphatase conjugate antibody; and the like. Additional labels of interest include those that provide for signal only when the nucleic acid with which they are associated is specifically bound to perfectly complementary nucleic acid molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037; WO 97/17471 and WO 97/17076.

Hybridization to Solid Supports

Hybridization refers to the formation of a biomolecular complex of two different nucleic acids through complementary base pairing. Complementary base pairing occurs through non-covalent bonding, usually hydrogen bonding of bases that specifically recognize other bases, as the bonding of complementary bases in double stranded DNA. In the present invention, hybridization is carried out between the amplified (labeled) nucleic acid and the complementary nucleic acids on the array. Those skilled in the art will appreciate that hybridization is not limited to Watson-Crick base-pairing interactions.

One skilled in the art will appreciate that an enormous number of nucleic acid array designs are suitable for the practice of the present invention. An array of nucleic acids will typically include a number of complementary nucleic acid molecules that specifically hybridize to the nucleic acid sequences amplified from the binding agent. It is preferred that an array include one or more control probes. In certain preferred embodiments, the array contains only a few, for example, 1-5 or 1-10 complementary nucleic acids. In other preferred embodiments, the array is a high-density array. A high-density array is an array used to hybridize with the amplified nucleic acids to detect the presence of a large number of target-binding agent complexes, for example, 10, 100, or even 1000 or more target-binding agent complexes.

The array of complementary nucleic acids can be arranged on a support in a pattern according to their identity, i.e., according to which binding agent the complementary nucleic acid identifies (see, e.g., U.S. Pat. No. 6,287,768, incorporated herein by reference). The nucleic acids of the subject arrays are typically nucleotide base containing nucleic acids or at least mimetics or analogues of naturally occurring polymeric compounds. Biopolymeric compounds of particular interest are ribonucleic acids, as well as deoxyribonucleic acid derivatives thereof, generated through a variety of processes (usually enzymatic processes) such as reverse transcription, etc. As described herein, one particularly preferred embodiment utilizes reverse transcription to generate cDNA molecules from mRNA-protein binding agents (both single and double stranded). Of course, those skilled in the art will recognize that any of these processes may be carried out using one or more nucleic acid bases.

In the subject nucleic acid arrays, the complementary nucleic acids are preferably stably associated with the surface of a support. By stably associated is meant that the nucleic acids maintain their position relative to the support under hybridization and washing conditions. As such, the nucleic acids can be non-covalently or covalently stably associated with the support surface. Examples of non-covalent association include non-specific adsorption, specific binding through a specific binding pair member covalently attached to the support surface, and entrapment in a matrix material, e.g. a hydrated or dried separation medium, which presents the nucleic acid in a manner sufficient for binding, e.g. hybridization, to occur. Examples of covalent binding include covalent bonds formed between the nucleic acid and a functional group present on the surface of the support, e.g. —OH, where the functional group may be naturally occurring or present as a member of an introduced linking group, as described in greater detail below.

As mentioned above, the nucleic acid or protein array is typically present on a substrate. Certain substrates are rigid meaning that the support is solid and does not readily bend, i.e. the support is not flexible. Examples of solid materials, which are not rigid supports with respect to the present invention, include membranes, flexible plastic films, and the like. As such, rigid substrates are sufficient to provide physical support and structure to the nucleic acids present thereon under the assay conditions in which the array is employed, particularly under high throughput handling conditions. Other forms of solid supports include microparticles, beads, membranes, slides, plates, micromachined chips, micro-or macro-porous particles, microfluidic device and the like.

The substrates upon which the subject patterns of nucleic acids are preferably presented in the subject arrays may take a variety of configurations ranging from simple to complex, depending on the intended use of the array. Thus, the substrate could have an overall slide or plate configuration, such as a rectangular or disc configuration, where an overall rectangular configuration, as found in standard microtiter plates and microscope slides, is preferred. For example, the length of the substrates may be at least about 1 cm and may be as great as 40 cm or more, but usually does not exceed about 30 cm and may often not exceed about 15 cm. The width of substrate may be at least about 1 cm and may be as great as 30 cm, but usually does not exceed 20 cm and often does not exceed 10 cm. The height of the substrate will generally range from 0.01 mm to 10 mm, depending at least in part on the material from which the substrate is fabricated and the thickness of the material required to provide the requisite rigidity.

The substrates of the subject protein or nucleic acid arrays may be fabricated from a variety of materials. The materials from which the substrate is fabricated should ideally exhibit a low level of non-specific binding of amplified nucleic acid during hybridization or specific binding events. In many situations, it will also be preferable to employ a material that is transparent to visible and/or UV light. Specific materials of interest include: glass; plastics, e.g. polytetrafluoroethylene, polypropylene, polystyrene, polycarbonate, and blends thereof, and the like; metals, e.g. gold, platinum, and the like; etc.

The substrate of the subject arrays comprises at least one surface onto which a pattern of nucleic acid molecules is present, where the surface may be smooth or substantially planar, or have irregularities, such as depressions or elevations. The surface on which the pattern of nucleic acids is presented may be modified with one or more different layers of compounds that serve to modulate the properties of the-surface in a desirable manner. Such modification layers, when present, will generally range in thickness from a monomolecular thickness to about 1 mm, usually from a monomolecular thickness to about 0.1 mm and more usually from a monomolecular thickness to about 0.001 mm. Modification layers of interest include: inorganic and organic layers such as metals, metal oxides, polymers, small organic molecules and the like. Polymeric layers of interest include layers of: peptides, proteins, polynucleic acids or mimetics thereof, e.g. peptide nucleic acids and the like; polysaccharides, phospholipids, polyurethanes, polyesters, polycarbonates, polyureas, polyamides, polyethyleneamines, polyarylene sulfides, polysiloxanes, polyimides, polyacetates, and the like, where the polymers may be hetero- or homopolymeric, and may or may not have separate functional moieties attached thereto, e.g. conjugated.

The concentration of the nucleic acid positions on the surface of the support is selected to provide for adequate sensitivity of binding events with the amplified nucleic acid, where the concentration will generally range from about 1 to 100, usually from about 5 to 50 and more usually from about 10 to 30 ng/mm². As summarized above, the subject arrays comprise a plurality of different complementary nucleic acids or sets of nucleic acids, where the number of nucleic acids is at least 5, usually at least 8, and may be much higher. In some embodiments, the arrays have at least 10 distinct spots, usually at least about 20 distinct spots, and more usually at least about 50 distinct spots, where the number of spots may be as high as 10,000 or higher, but will usually not exceed about 5,000 distinct spots, and more usually will not exceed about 3,000 distinct spots. The density of the spots on the solid surface in certain embodiments is at least about 5/cm² and usually at least about 10/cm² but does not exceed about 1000/cm², and usually does not exceed about 500/cm², and more usually does not exceed about 300/cm².

The protein and nucleic acid arrays of the subject invention may be used directly in binding assays, i.e., hybridization assays, using well known technologies, e.g. contacting with amplified nucleic acid in a suitable container, under a coverslip, etc, or may be incorporated into a structure that provides for ease of analysis, high throughput, or other advantages, such as in a biochip format, a multiwell format and the like. For example, the subject arrays could be incorporated into a biochip type device in which one has a substantially rectangular shaped cartridge comprising fluid entry and exit ports and a space bounded on the top and bottom by substantially planar rectangular surfaces, wherein the array is present on one of the top and bottom surfaces.

Alternatively, the subject protein and nucleic acid arrays may be incorporated into a high throughput or multiwell device, wherein each array is bounded by raised walls in a manner sufficient to form a reaction container wherein the array is the bottom surface of the container. Such high throughput devices are described in U.S. patent application Ser. No. 08/974,298, now abandoned, the disclosure of which is herein incorporated by reference. Generally in such devices, the devices comprise a plurality of reaction chambers, each of which contains the array on the bottom surface of the reaction chamber. By plurality is meant at least 2, usually at least 4 and more usually at least 24, where the number of reaction chambers may be as high as 96 or higher, but will usually not exceed 100. The volume of each reaction chamber may be as small as 10 μl but will usually not exceed 500 μl.

Representative fluorescence detection devices include the Affymetrix GeneArray Scanner (Affymetrix, Santa Clara, Calif.) and Axon GenePix 4000™ microarray scanner (Axon Instruments, Foster City, Calif.). Also of interest are nanometer sized particle labels detectable by light scattering, e.g. “quantum dots.”

The subject protein and nucleic acid arrays may be prepared as follows. The substrate or support can be fabricated according to known procedures, where the particular means of fabricating the support will necessarily depend on the material from which it is made. For example, with polymeric materials, the support may be injection molded, while for metallic materials, micromachining may be the method of choice. Alternatively, supports such as glass, plastic, or metal sheets can be purchased from a variety of commercial sources and used. The surface of the support may be modified to comprise one or more surface modification layers, as described above, using standard deposition techniques.

Typically, the next step in the preparation process is to prepare the pattern of nucleic acid molecules and then stably associate the nucleic acid molecules with the surface of the support. The nucleic acids may be deposited on the support surface using any convenient means, such as by using an “ink-jet” device, mechanical deposition, pipetting and the like. After deposition of material onto the solid surface, it can be treated in different ways to provide for stable association of the nucleic acid, blockage of non-specific binding sites, removal of unbound nucleic acid, and the like.

Following stable placement of the pattern of nucleic acid (or protein) molecules on the support surface, the resultant array may be used as is or incorporated into a biochip, multiwell or other device, as described above, for use in a variety of binding applications.

The subject protein or nucleic acid arrays or devices into which they are incorporated may conveniently be stored following fabrication for use at a later time. Under appropriate conditions, the subject arrays are capable of being stored for at least about 6 months and may be stored for up to one year or longer. The subject arrays are generally stored at temperatures between about −20° C. to room temperature, where the arrays are preferably sealed in a plastic container, e.g. bag, and shielded from light.

The length of the complementary nucleic acid will generally range from about 10 to 2000 nucleotides, where oligonucleotides will generally range in length from about 15 to 100 nucleotides and polynucleotides will generally range in length from about 100 to 1000 nucleotides, where such probes may be single or double stranded, but will usually be single stranded.

The next step in the subject method is to contact the amplified nucleic acids with the complementary nucleotides on the array under conditions sufficient for binding between the amplified nucleic acid and the complementary nucleic acid of the array. For example, the amplified nucleic acid will be contacted with the array under conditions sufficient for hybridization to occur between the amplified nucleic acid and the complementary nucleic acid, where the hybridization conditions will be selected in order to provide for the desired level of hybridization specificity.

Contact of the array and amplified nucleic acid involves contacting the array with an aqueous medium comprising the amplified nucleic acid. Contact may be achieved in a variety of different ways depending on the specific configuration of the array. For example, where the array simply comprises the pattern of complementary nucleic acids on the surface of a “plate-like” substrate, contact may be accomplished by simply placing the array in a container comprising the amplified nucleic acid solution, such as a polyethylene bag, small chamber, and the like. In other embodiments where the array is entrapped in a separation media bounded by two plates, the opportunity exists to deliver the amplified nucleic acid via electrophoretic means. Alternatively, where the array is incorporated into a biochip device having fluid entry and exit ports, the amplified nucleic acid solution can be introduced into the chamber in which the pattern of nucleic acid molecules is presented through the entry port, where fluid introduction could be performed manually or with an automated device. In multiwell embodiments, the amplified nucleic acid solution will be introduced in the reaction chamber comprising the array, either manually, e.g. with a pipette, or with an automated fluid handling device.

Contact of the amplified nucleic acid solution and the complementary nucleic acids will be maintained for a sufficient period of time for binding between the amplified nucleic acid and the complementary nucleic acid to occur. Although dependent on the nature of the amplified nucleic acid and complementary nucleic acid, contact will generally be maintained for a period of time ranging from about 10 min to 24 hrs, usually from about 30 min to 12 hrs and more usually from about 1 hr to 6 hrs.

Following binding of amplified nucleic acid and complementary nucleic acid, the resultant hybridization patterns of labeled amplified nucleic acids may be visualized or detected in a variety of ways, with the particular manner of detection being chosen based on the particular label of the amplified nucleic-acid, where representative detection means include, e.g., scintillation counting, autoradiography, fluorescence measurement, colorimetric measurement, light emission measurement and the like.

The method may or may not further include a non-bound label removal step prior to the detection step, depending on the particular label employed on the amplified nucleic acid. For example, in homogenous assay formats a detectable signal is only generated upon specific binding of amplified nucleic acid to complementary nucleic acid. As such, in homogenous assay formats, the hybridization pattern may be detected without an unbound amplified nucleic removal step. In other embodiments, the label employed will generate a signal whether or not the amplified nucleic acid is specifically bound to its complementary nucleic acid. In such embodiments, the unbound labeled amplified nucleic acid sample is removed from the support surface. One means of removing the unbound labeled amplified nucleic acid is to perform the well known technique of washing, where a variety of wash solutions and protocols for their use in removing unbound label are known to those of skill in the art and may be used. Alternatively, in those situations where the nucleic acids are entrapped in a separation medium in a format suitable for application of an electric field to the medium, the opportunity may arise to remove unbound labeled amplified nucleic acid from the complementary nucleic acid by electrophoretic means.

Kits

The present invention further provides kits for identifying and quantifying protein-protein interactions. The kit includes one or more capture agents, one or more binding agents that include a unique protein portion and a unique nucleic acid sequence (e.g., RNA, DNA, or mRNA), and preferably an array of nucleic acids, which are complementary to the nucleic acid sequences on the binding agents.

In certain preferred embodiments, an inventive kit provides binding agents that are labeled. According to certain preferred embodiments, the nucleic acid sequence that is fused to the binding agent is labeled. As stated herein, any detectable label, such as a fluorescent label, radioactive label, or mass tag label, etc., may be included in the kit for labeling the nucleic acid portion of the binding agent or already bound to the binding agent. For the convenience of the user, the kit may also include reagents for amplifying the nucleic acid portion of the binding agent. The amplifying reagents may further include labeled and non-labeled primers for amplifying the nucleic acids. One or both of the forward and reverse primers provided in the kit may be complementary to the unique region of the nucleic acid portion or to a region of the nucleic acid portion that is common to all nucleic acids. Reagents for detecting the label on the amplified nucleic acid may also be included in the kit, e.g., horseradish peroxidase detection system reagents. Alternatively, the nucleic acids fused to the binding agents contain detectable labels that can be detected directly, without amplification.

In preferred embodiments, inventive kits provide capture agents that are attached to a solid support, e.g., a column, a well, a plate, a slide etc., such as are known in the art. In one preferred embodiment, the capture agents provided in the kits are attached to magnetic beads. The kits of the invention may thus include capture agents that are specific to particular targets and are attached to one or more solid supports.

The present invention is demonstrated by the following non-limiting examples.

### EXEMPLIFICATION

### EXAMPLE 1

Magnetic Bead Format with mRNA-Protein Binding Agent

In the present Example, the capture agent (C1) is attached to a magnetic bead. For multiplexing, different capture agents (C1, C2 . . . Cn) are attached to separate or the same magnetic beads. A complex sample, containing multiple target proteins (T1, T2 . . . Tn) is added to a suspension of the beads in an appropriate buffer and allowed to bind. Each of the target proteins is likely to be present in different concentrations as low as zero (target protein not present). After binding of the target to the capture agent, the magnetic beads are separated from the solution (using a magnet) and washed with appropriate buffers to remove irrelevant chemical species. The beads are then resuspended and a mixture of binding agents (B1, B2 . . . Bn) is added and allowed to bind to the appropriate target proteins. Each of the secondary binding agents is fused to an mRNA nucleic acid sequence (N1, N2 . . . Nn) that either is a simple unique identifier or is a result of the translation of the binding agent itself.

Once the binding agent is bound to the preformed capture agent-target protein complex, the beads are separated from solution and washed to remove unmatched binding agent. At this point, the mRNA sequence is amplified directly from the complex or cleaved and then amplified in solution. The amplification is carried out with a labeled primer so that each amplified sequence generates a specific signal intensity. Assuming that all sequences amplify equally, the concentration of the original target population is represented proportionately and geometrically multiplied.

Following amplification, the nucleic acid population is separated from the capture agent-target-binding agent complex. This is achieved by removing from the solution the magnetic beads to which the capture agent-target-binding agent complex is attached. The amplified nucleic acids are then applied to a DNA array in each feature containing the complimentary sequence for the individual unique identifier sequence in the amplified sample.

The DNA arrays are processed in a standard manner including the steps of: hybridizing the amplified nucleic acid to the complementary nucleic acids on the array; washing-the unbound amplified nucleic acid from the hybridized nucleic acid, imaging the bound (labeled) amplified nucleic acid by confocal fluorescence scanning, and analyzing by the same software used for DNA array applications. The resulting concentration of each identifier nucleic acid (“identifier” nucleic acid referring to the amplified nucleic acid) is used to calculate the original concentration of the target protein. For n target proteins, the requirements of this method are at least n high affinity capture agents bound to magnetic beads, n binding agents consisting of a high affinity binder directed at a free site and a fused mRNA sequence (consequently n mRNA sequences are also required), and one pair of labeled forward and reverse primers that are capable of amplifying the mRNA (before or after reverse transcription).

### EXAMPLE 2

Identification of Binding Agents

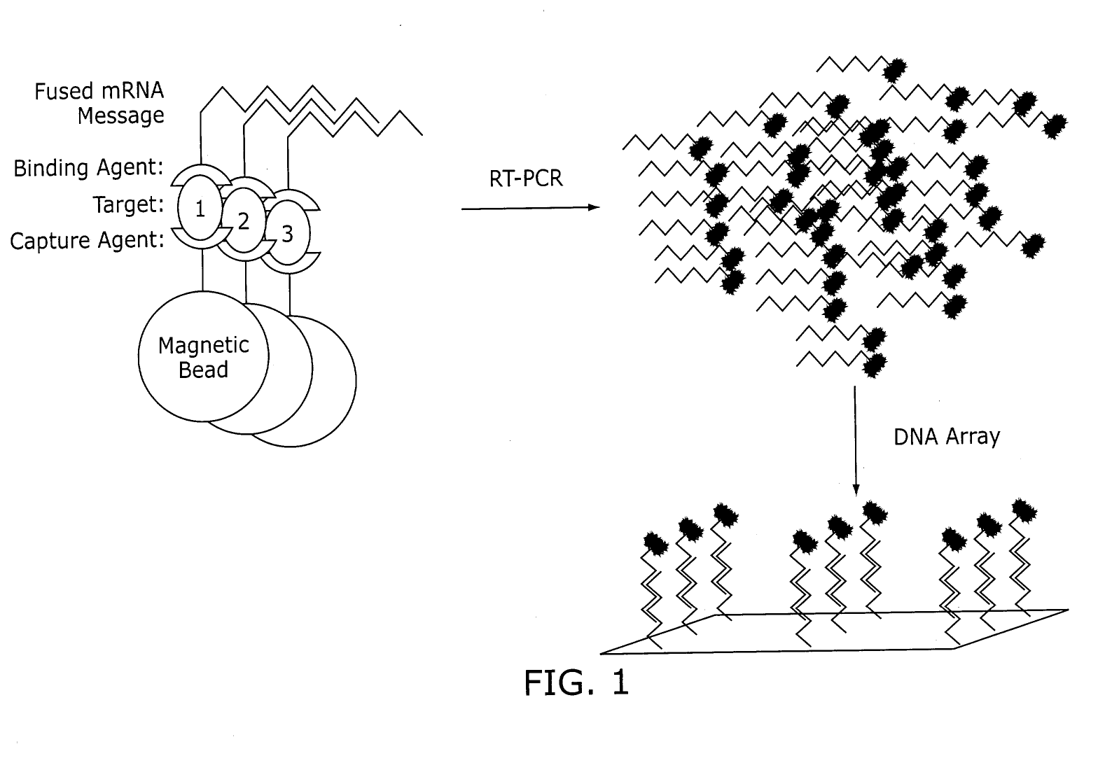

As shown in FIG. 1, capture agents (C1, C2 . . . Cn) generated against targets (T1, T2 . . . Tn) are immobilized on magnetic beads. A target protein sample, e.g., a complex biological sample or substantially purified protein sample, is contacted with the capture agent for a time and under conditions sufficient for binding of the target protein to the capture agent to occur. Unbound sample components are washed away using standard techniques and binding agents (B1, B2 . . . Bn), having fused nucleic acid portions (N1, N2 . . . Nn) are added and allowed sufficient time to bind the target protein before the unbound binding agents are washed away. Wherein the nucleic acid portion is mRNA, the mRNA portion of the binding agent is then amplified by RT-PCR and analyzed using DNA array technology. Alternatively, if the amplified nucleic acids include cleavable mass tags, the nucleic acids are analyzed using APCI-MS.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

Each reference cited herein is hereby incorporated by reference.

We claim:

1. A method for detecting a protein-protein interaction, the method comprising the steps of:
providing one or more capture agents attached to a solid support;
contacting the capture agent with one or more target proteins for a time sufficient to allow binding of the target protein to the capture agent to form capture agent-target complex;
removing unbound target from a capture agent-target complex;
contacting the capture agent-target complex with at least one binding agent capable of binding to the target protein, wherein the binding agent binds to a different site on the target than the capture agent, wherein the binding agent comprises a protein portion and a nucleic acid portion, wherein the nucleic acid portion is unique to the binding agent;
amplifying the nucleic acid portion of the binding agent;
applying the amplified nucleic acid portion to an array of complementary nucleic acids and allowing the amplified nucleic acid portions to hybridize with the complementary nucleic acids on the array;
removing unbound amplified nucleic acid portions from the array;
detecting the bound amplified nucleic acid portions on the array.

2. The method of claim 1, wherein each nucleic acid portion has a different nucleic acid sequence.

3. The method of claim 1, wherein the binding agent is identified by its location on the spatial array of complementary nucleic acids.

4. The method of claim 1, further comprising quantifying the amplified nucleic acid on the array.

5. The method of claim 4, wherein the quantity of the amplified nucleic acid is proportional to the quantity of the target.

6. The method of claim 1, wherein the amplified nucleic acid portion comprises a detectable label.

7. The method of claim 6, wherein in the step of amplifying the nucleic acid portion, a detectable label is incorporated into the nucleic acid portion during the amplification process.

8. The method of claim 6, wherein a detectable label is incorporated into the nucleic acid portion using a labeled primer during amplification.

9. The method of claim 1, wherein different capture agents are attached to different solid supports.

10. The method of claim 1, wherein different capture agents are attached to the same solid supports.

11. The method of claim 1, wherein the array comprises a plurality of nucleic acids, wherein at least a portion of the nucleic acids are complementary to the amplified nucleic acid portions.

12. The method of claim 1, wherein the nucleic acid portion comprises an mRNA.

13. The method of claim 12, wherein the binding agent is a translation product of the mRNA nucleic acid portion of the binding agent.

14. The method of claim 1, wherein the nucleic acid portion comprises DNA.

15. A method for quantifying a target protein, the method comprising the steps of: providing one or more capture agents attached to the surface of a solid support; contacting the capture agents with one or more target proteins for a time sufficient to allow binding of the target protein to the capture agent to form capture agent-target complex;
removing unbound targets from a capture agent-target complex;
contacting the capture agent and the target protein with at least one binding agent that is capable of binding to a corresponding target protein, wherein the binding agent binds to a different site on the target than the capture agent, wherein the binding agent comprises a protein portion and an nucleic acid portion that is unique to the binding agent;
amplifying the nucleic acid portion of the binding agent, wherein the amplified nucleic acid comprises a detectable label;
applying the amplified nucleic acid portions to an array of complementary nucleic acids and allowing the amplified nucleic acid portions to hybridize to the complementary nucleic acids on the array;
removing unbound amplified nucleic acid portions from the array;
quantifying the bound amplified nucleic acid portions on the array, wherein the quantity of the amplified nucleic acid indicates that quantity of the target.

16. The method of claim 15, wherein in the step of amplifying the nucleic acid portions, a detectable label is incorporated into the nucleic acid portion during the amplification process.

17. The method of claim 15, wherein a detectable label is incorporated into the amplified nucleic acid portion by using a labeled primer during amplification.

18. The method of claim 15, wherein different capture agents are attached to different solid support.

19. The method of claim 15, wherein different capture agents are attached to the same solid support.

20. The method of claim 15, wherein the nucleic acid portion comprises mRNA.

21. The method of claim 20, wherein the protein portion is a translation product of the mRNA nucleic acid portion.

22. The method of claim 15, wherein the amplified nucleic acid portion comprises DNA.

23. A method of detecting a binding agent that is bound to a target protein, the method comprising the steps of:
providing one or more capture agents attached to a solid support in a spatial arrangement, wherein the position of the capture agent identifies the capture agent;
contacting the capture agents with one or more target proteins for a time sufficient to allow binding of the target protein to the capture agent to form a capture agent-target protein complex;
removing unbound target from the capture agent-target complex;
contacting the capture agent-target complex with at least one binding agent capable of binding to the target, wherein the binding agent binds to a different site on the target than the capture agent, wherein the binding agent comprises a protein portion and a nucleic acid portion comprising a unique label;
detecting the unique label, thereby identifying the binding agent; and
detecting the location of capture agent, thereby identifying the capture agent.

24. A kit for detecting or quantifying protein-protein interactions comprising:
one or more capture agents, wherein the capture agent is capable of binding to a first site on a target protein;
one or more binding agents, wherein the binding agent binds to a second site on the target, wherein the binding agent comprises a protein portion and a nucleic acid portion, wherein the nucleic acid portion is unique to the binding agent;
an array of nucleic acids that are complementary to the nucleic acid portion of the binding agents.

25. The kit of claim 24, wherein the nucleic acid portion comprises a detectable label.

26. The kit of claim 24, further comprising reagents for amplifying the nucleic acid portion.

27. The kit of claim 26, wherein the reagents for amplifying the nucleic acid portion further comprise the ability to label the nucleic acid portion with a detectable label.

28. The kit of claim 24, further comprising reagents for detecting the detectable label.

29. The kit of claim 24, wherein the capture agent is on a solid support.

30. The kit of claim 29, wherein different capture agents are attached to different solid supports.

31. The kit of claim 29, wherein different capture agents are attached to the same solid support.

32. The kit of claim 24, wherein the nucleic acid portion comprises mRNA.

34. The kit of claim 24, wherein the nucleic acid portion comprises DNA.
